(19) United States
(12) Patent Application Publication
(10) Pub. No.: US 2015/0204853 A1
(43) Pub. Date: Jul. 23, 2015

(54) MODULATORS OF ION TRANSPORT PROTEINS OR ION CHANNELS AND METHODS OF IDENTIFYING AND USING SAME

(71) Applicant: Turfts University, Boston, MA (US)

(72) Inventors: Stephen J. Moss, Dover, MA (US); Tarek G. Deeb, Boston, MA (US)

(73) Assignee: TUFTS UNIVERSITY, Boston, MA (US)

(21) Appl. No.: 14/148,699

(22) Filed: Jan. 6, 2014

(51) Int. Cl. G01N 33/50; C07K 16/18; G01N 33/58; A61K 31/7088

(52) U.S. Cl. CPC G01N 33/5041; A61K 31/7088; C07K 16/18; G01N 33/582; G01N 2500/04; G01N 2500/10

(57) ABSTRACT

Described herein are materials and methods for measuring intracellular ion concentrations, materials and methods for identifying modulators of intracellular ion concentrations, modulators if intracellular ion concentration, use of modulators of intracellular ion concentration to treat a disease or disorder associated with aberrant intracellular ion concentrations, and cell lines and transgenic animals that express a fluorescent ion detector and/or one or more ion transport proteins or ion channels.

### RELATED APPLICATIONS

This application claims the benefit of U.S. provisional application No. 61/749,459, filed Jan. 7, 2013, the entire contents of each of which are herein incorporated by reference.

### GOVERNMENT SUPPORT

This invention was made with government support under grant 047478 awarded by the National Institute of Neurological Disorders and Stroke (NINDS). The government has certain rights in the invention.

### BACKGROUND

Cl⁻ is a major constituent of cells and extracellular compartments. Cl⁻ transport across cell membranes is crucial to many key cellular processes such as cell volume and pH regulation, transepithelial fluid transport and synaptic transmission. Within cells, Cl⁻ transport is involved in organellar acidification. A variety of human diseases are associated with mutations in Cl⁻ transporting proteins.

Despite the importance of Cl⁻ for many cellular functions, there is little knowledge available on the mechanisms regulating Cl⁻ in either physiological or pathological conditions. This lack of information is primarily due to technical difficulties in the measurement of Cl⁻ flux in live cells.

### SUMMARY

Described herein are methods for measuring ion concentration, e.g., Cl⁻, methods of screening for molecules and compounds that modulate ion transport proteins or ion channels, e.g., KCC2 and/or NKCC1, compositions and methods of using modulators of ion transport proteins or ion channels, e.g., for use in treating diseases or disorders associated with, for example, misregulation of KCC2 and/or NKCC1 and cell lines and transgenic animals that express Clomeleon and/or one or more exogenous ion transport proteins or ion channels.

In one embodiment, the disclosure is directed to a method of identifying a modulator of an ion transport protein or ion channel comprising, a) contacting a cell line that expresses a fluorescent ion detector with a test agent; b) creating a chloride challenge to the contacted cell line; and c) determining the intracellular chloride ion concentration, wherein an intracellular chloride ion concentration that is statistically different from a cell line not contacted with the test agent and exposed to the same chloride ion challenge indicates that the test compound is a modulator of an ion transport protein or ion channel. In a particular embodiment, the ion channel/transporter is a chloride ion channel/transporter. In a particular embodiment, the chloride ion channel/transporter is KCC2 or NKCC1. In a particular embodiment, the fluorescent ion detector is Clomeleon. In a particular embodiment, the identified modulator is further validated in a transgenic animal. In a particular embodiment, the transgenic animal expresses a fluorescent ion detector. In a particular embodiment, the fluorescent ion detector is Clomeleon.

In one embodiment, the disclosure is directed to a modulator of an ion transport protein or ion channel that is identified by any of the methods described herein. The modulator can be used, for example for the treatment of a disease or disorder associated with misregulated ion transport. The modulator can be used, for example, to make a medicament for the treatment of a disease or disorder associated with misregulated ion transport.

In one embodiment, the disclosure is directed to a method of treating a disease or disorder associated with aberrant intracellular ion concentrations comprising administering a therapeutically effective amount of a modulator identified by any of the methods described herein.

In one embodiment, the disclosure is directed to an engineered cell line comprising: a) a vector that expresses a fluorescent ion detection protein; and b) a vector that expresses an exogenous ion transport protein or ion channel. In a particular embodiment, the fluorescent ion detection protein is Clomeleon. In a particular embodiment, the exogenous ion transport protein or ion channel is KCC2 or NKCC1. In a particular embodiment, the cell line is a human embryonic kidney cell line, a Chinese hamster lung cell line, a COS-7 cell line or a fibroblast cell line.

In one embodiment, the disclosure is directed to a transgenic animal comprising a vector comprising a fluorescent ion detection protein. In a particular embodiment, the transgenic animal further comprises a vector that expresses an exogenous ion transport protein or ion channel. In a particular embodiment, the animal is a mammal. In a particular embodiment, the mammal is a rodent.

### BRIEF DESCRIPTION OF THE DRAWINGS

FIGS. 1A and B indicate Cl⁻ homeostasis is determined by cation-chloride transporters. FIG. 1A is a scheme of Cl⁻ homeostasis when NKCC1 is dominant. Glycine and GABA_{A} currents are outward, depolarizing and excitatory. FIG. 1B is a scheme of Cl⁻ homeostasis when KCC2 is dominant. Glycine and GABA_{A} currents are inward, hyperpolarizing and inhibitory.

FIG. 2 shows an assay in a cell expressing KCC2, Clomeleon and Cl⁻ channels. Left, at rest KCC2 generates a low Cl⁻ concentration inside the cell, resulting in a high Clomeleon (CHL) fluorescence signal. Center, activation of a glycine receptor (GIyR) or halorhodopsin (HLR) causes a massive Cl⁻ load and decreases Clomeleon fluorescence. Right, termination of the Cl⁻ loading phase allows the cell to reestablish low Cl⁻ levels resulting in increasing Clomeleon fluorescence over time.

FIGS. 3A and B show a single unit of OCTA cellular and solutions chamber. FIG. 3A is a schematic of one removable cellular growth unit. FIG. 3B shows two adjacent units. The cellular units can be slotted into position between inlet and outlet tubes fixed to the imaging platform. The inlet tube divides into three separate tubes, with each connected to its own reservoir (not shown) containing appropriate solutions. The outlet tube can be connected to a vacuum trap for waste collection. Arrows indicate the direction of solution flow.

### DETAILED DESCRIPTION

Described herein are materials and methods for detecting intracellular chloride ion concentrations. The disclosure is also directed to methods for identifying compounds that alter the intracellular chloride ion concentration, for example, by modulating the activity of ion transporters and/or ion channels. The disclosure, for example, can be used to identify activators of particular chloride transporters, e.g., KCC2 or NKCC1, to ameliorate or prevent a pathological state in a subject. The disclosure is further directed to compositions that modulate ion transport proteins and/or ion channels and methods of using such compositions for treating diseases or disorders associated with misregulated ion transport or aberrant cellular ion concentrations. The disclosure is further directed to cell lines and transgenic animals that express, for example, a fluorescent ion detector, e.g., Clomeleon (Berglund, K., Kuner, T. & Augustine, G., “Clomeleon, a genetically encoded chloride indicator.” In: Alvarez-Leefmans, F. & Delpire, E. (eds) Physiology and pathology of chloride transporters and channels in the nervous system, London, UK. Elsevier, 125-139, 2009), and/or one or more exogenous ion transport proteins or ion channels, and/or one or more proteins that inducibly create an ion challenge to a cell.

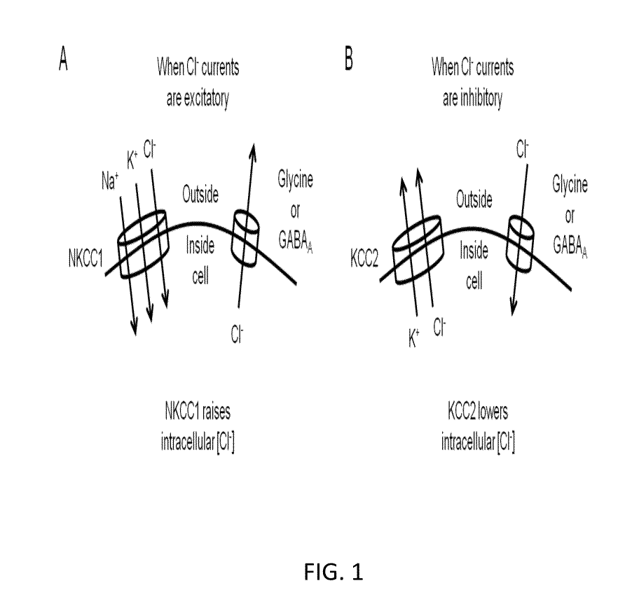

Chloride ion transport in cells, including engineered cell lines as described herein or mammalian neurons, is determined by the concerted activity of cation-chloride transporters such as, for example, KCC2 or NKCC1, which either extrude Cl⁻ from the cell interior or load Cl⁻ into the cell, respectively (FIG. 1). These processes allow the proper functioning of neurons, as Cl⁻ is the third most abundant permeable ion in the human body and responsible for the proper functioning of several biological processes. Cl⁻ produces, for example, the predominant synaptic inhibitory current mediated by GABA_{A} and glycine receptors in the nervous system. Misregulation of the Cl⁻ homeostatic mechanism is routinely observed in several neurological disorders, with a shift from a KCC2-dominated system to one that lacks a strong Cl⁻ extrusion mechanism. This situation results in a loss of GABAergic and glycinergic inhibitory signaling and a decrease in the therapeutic efficacy of drugs that target the GABA_{A} and/or glycine receptors, which includes the benzodiazepine class of drugs (e.g., Valium), benzodiazepine-related compounds (e.g., Ambien) and anesthetics such as, for example, propofol and barbiturates. There currently are few assays to measure chloride transport, and none that utilize a high-throughput approach using neurons.

Described herein is an optical cation-chloride transporter assay (OCTA), which is a fluorescence-based, real-time assay for cation-chloride transporter activity. OCTA can be used, for example, to discover compounds that increase or restore KCC2 function and increase the efficacy of medications. OCTA is used to discover entirely new compounds or assay combinations of compounds, including more selective modulators of NKCC1. The widely used loop-diuretic bumetanide, for example, is being used to treat neonatal seizures; however, bumetanide can also inhibit GABA_{A} receptors. More selective inhibitors would increase the therapeutic efficacy of this strategy, while reducing side effects. OCTA is also used to discover selective KCC2 modulators, e.g., as diuretics, therapeutics or for research purposes.

As used herein, the term “modulator” refers to a molecule, compound or composition that modulates one or more physiological or biochemical events. In some embodiments the modulator inhibits one or more biological activities. In other embodiments the modulator increases one or more biological activities. In some embodiments the modulator is a small molecule, an antibody, a mimetic, a decoy or an oligonucleotide. In some embodiments the modulator acts by blocking ligand binding or by competing for a ligand-binding site. In some embodiments the modulator acts independently of ligand binding. In some embodiments the modulator does not compete for a ligand binding site. In some embodiments the modulator blocks expression of a gene product. In some embodiments the modulator blocks a physical interaction of two or more biomolecules.

As used herein, “chloride” refers to the charged ion form (Cl⁻) of chlorine. The concentration of chloride within a cell, i.e., the intracellular chloride concentration, can also be referred to as a chloride level or intracellular chloride level.

As used herein, “KCC” refers to any of the K⁺-CL⁻ contransporter proteins. The predominant member of the KCC family of proteins is KCC2 (potassium-chloride transporter member 5; SLC12A5), which is a neuron-specific chloride potassium symporter responsible for establishing the chloride ion gradient in neurons through the maintenance of low intracellular chloride concentrations. Unless specifically stated, “KCC2” refers to either of the two KCC2 isoforms, KCC2a and KCC2b.

KCC2 is a neuron specific cation-chloride cotransporter that performs K—Cl extrusion, which leads to GABA_{A}- and glycine receptor mediated inhibitory hyperpolarizing chloride currents. Without KCC2 function, intracellular chloride levels rise, resulting in depolarizing and in some cases excitatory GABAergic and glycinergic responses. KCC2 is therefore analogous to an endogenous break on excitability, and without this break, the nervous system progresses into a state of hyperexcitability and aberrant signaling.

In humans, KCC2 is downregulated in resected brain tissue from patients suffering from temporal lobe epilepsy (Payne, J. et al., Trends Neurosci., 26:199-206, 2003; Galanopoulou, A., Epilepsia, 48 Suppl. 5:14-8, 2007; Kahle, K. et al., Nat. Clin. Pract. Neurol., 4:490-503, 2008; Blaesse, P. et al., Neuron, 61:820-38 2009; Loscher, W. et al., Neuropharmacology, 69:62-74, 2013) and brain tumors (Wu, J. et al., Exp. Neurol., 213:397-404, 2008; Conti et al., 2011). Reduced KCC2 function that leads to aberrant GABAergic and/or glycinergic signaling has also been demonstrated in several animal models including chronic pain (Zhou, H. et al., J. Biol. Chem., 287:33853-64, 2012), acute stress (Sarkar, J. et al., J. Neurosci., 31:18198-210, 2011), stroke (Jaenisch, N. et al., Stroke, 41:e151-9, 2010), peripheral nerve damage (Hasbargen, T. et al., Ann. NY Acad. Sci., 1198:168-72, 2010), amyotrophic lateral sclerosis (Fuchs, A. et al., J. NeuropathoL Exp. Neurol., 69:1057-70, 2010) and seizures. Loss of KCC2 function is a common occurrence in many nervous system disorders in animal and human disorders, including, for example, Alzheimer's and traumatic brain injury. Restoration of KCC2 function is, therefore, important to prevent or ameliorate pathological conditions associated misregulation of KCC2 activity.

In addition to changes in KCC2 mRNA and total protein levels, changes in the surface levels and transporter activity, i.e., surface activity, are important (Chamma, I. et al., Front. Cell Neurosci., 6:5, 2012). Phosphorylation sites on KCC2 regulate both the trafficking of KCC2 to and from the membrane, as well as its transporter activity. The two major kinases that are known to directly phosphorylate KCC2 residues are the serine/threonine kinases from the WNK (Rinehart, J. et al., Cell, 138:525-36, 2009) and PKC families (Lee, H. et al., J. Biol. Chem., 282:29777-84, 2007; Lee, H. et al., Nat. Neurosci., 14:736-43, 2011). Wnk1/Wnk3 and tyrosine kinase (i.e., TrkB) phosphorylation downregulates KCC2 activity. Whereas WNK phosphorylation decreases KCC2 function, PKC increases surface activity. Many G-protein-coupled receptors (GPCRs), for example, are known to couple through the PLC/PKC pathway. PKC phosphorylation of the C-terminus Ser940 residue of the KCC2 protein upregulates KCC2 activity by increasing surface stability (Blaesse, P. et al., Neuron, 61:820-38, 2009). This, combined with the observation that the KCC2 PKC site is basally phosphorylated throughout the brain, indicates there are basally active endogenous mechanisms supporting KCC2 function that can be utilized by therapeutic intervention.

KCC2 is downregulated by excitatory glutamate activity on NMDA receptor activity and Ca²⁺ influx (Ginsberg, M., Neuropharmacology, 55:363-89, 2008). KCC2 is transcriptionally downregulated following central nervous system injury by the TrkB receptor signaling transduction cascade (activated by BDNF and NT-4/5)(Rivera, C. et al., J. Cell Biol., 159:747-52, 2002; Rivera, C. et al., J. Neurosci., 24:4683-91, 2004; Kovalchuk, Y. et al., Curr. Opin. Neurobiol., 14:558-63, 2004).

As used herein, “NKCC” refers to any of the Na—K—Cl cotransporter proteins. NKCC (SLC12A2) is a protein that aids in the active transport of sodium, potassium and chloride into and out of cells. There are two varieties, or isoforms, of this membrane transport protein, called NKCC1 and NKCC2. NKCC1 is widely distributed throughout the body; it has important functions in organs that secrete fluids. NKCC2 is found specifically in the kidney, where it serves to extract sodium, potassium, and chloride from the urine so that they can be reabsorbed into the blood.

Methods have been proposed and used for monitoring intracellular chloride concentration. Early approaches with chloride-selective microelectrodes showed that the intracellular chloride concentration in some giant neurons of the snail Helix aspersa is low, about 8 mM (Neild, T. & Thomas, R., J. Physiol., 242:453-70, 1974). This method has been used in several cell types, including astrocytes, mouse lacrimal acinar cells (Ozawa, T. et al., Pflugers Arch., 412:509-15, 1988) and horizontal cells of the fish retina (Djamgoz, M. & Laming, P., Vision Res., 27:711-21, 1987). These chloride-selective electrodes, however, can only be reliably applied to relatively large cells.

Dysfunction of chloride ion channels and transporters or misregulation of intracellular chloride ion concentration results in diseases such as, for example, Bartter's syndrome, startle disease, cystic fibrosis, ischemia, auditory disorders and epilepsy (Alvarez-Leefmans, F. & Delpire, E. (eds.) Physiology and pathology of chloride transporters and channels in the nervous system. London, UK. Elsevier; 111-123, 2009; Ashcroft, F., Ion channels and disease: channelopathies. London, UK. Academic Press; 2000.; Lerche, H. et al., Curr. Pharm. Des., 11:2737-52, 2005). Dent's disease, renal salt wasting, hypokalemic alkosis, low molecular weight proteinuria, hypercalciuria, Thomsen disease and Becker disease have also been associated with misregulated chloride channels leading to pathological intracellular chloride levels.

The methods described herein can be used, for example, to identify modulators of intracellular chloride ion concentration. Such modulators, for example, can modulate the activity of, for example, ion transport proteins and/or ion channels (either directly or indirectly though, for example, transcriptional regulations). The use of modulators identified herein would be directed by one of skill in the art to treat diseases or disorders associated with aberrant intracellular ion concentrations.

“Treatment” refers to the administration of a therapeutic agent or the performance of medical procedures with respect to a patient or subject, for either prophylaxis (prevention) or to cure or reduce the symptoms of a disease or disorder. The compounds described herein or identified through methods described herein can be used as part of a treatment regimen in therapeutically effective amounts. A “therapeutically effective amount” is an amount sufficient to decrease, prevent or ameliorate the symptoms associated with a medical condition.

The terms “patient” and “subject” mean all mammals including humans.

The treatment(s) described herein are understood to utilize formulations including compounds identified herein or identified through methods described herein and, for example, salts, solvates and co-crystals of the compound(s). The compounds of the present disclosure can exist in unsolvated as well as solvated forms with pharmaceutically acceptable solvents such as, for example, water, ethanol, and the like. In general, the solvated forms are considered equivalent to the unsolvated forms for the purposes of the present disclosure.

Methods described herein to measure intracellular ion concentrations can involve the expression of a fluorescent fusion protein that detects ion concentration, e.g., halide ion concentration, e.g., chloride ion concentration. One example of such a fluorescent ion detector is Clomeleon-a fusion protein comprising cyan fluorescent protein (CFP) and the topaz variant of yellow fluorescent protein (YFP)(Kuner, T. & Augustine, G., Neuron, 27:447-59, 2000). The excitation of CFP (˜430-460 nm) causes the CFP subunit to emit at a wavelength that excites the nearby topaz YFP variant moiety. The construct utilizes the two fluorescent moieties to create a FRET excitation of the YFP moiety that allows one to measure baseline emission from the YFP moiety in a ratiometric fashion. As there is a cleavage site engineered between the two fluorescent moieties, the FRET excitation of the YFP moiety is eliminated upon cleavage and a baseline can be established by comparing emission from the YFP moiety before and after cleavage. The sensor function of Clomeleon is mediated by an intrinsic sensitivity of YFP to halides (Wachter, R. & Remington, S., Curr. Biol., 9:R628-9, 1999). Exposing YFP protein to solutions with increasing concentrations of Cl⁻ causes a reduction of YFP emission, while CFP emission remains largely unaffected.

Other ion indicators include, for example, Cameleon (Nagai, T. et al., Proc. Natl. Acad. Sci. USA, 101:10554-9, 2004).

Described herein are methods for determining intracellular ion concentrations. e.g., chloride, using cell lines and transgenic models that have incorporated a fluorescent chloride detector, e.g., Clomeleon, such detector having been permanently, transiently or inducibly incorporated into the cell line or transgenic animal. Expression of the fluorescent detector, e.g., Clomeleon, allows for the determination of chloride concentration by a fluorometric assay.

The methods described herein for the determination of chloride concentration can be used to identify modulators of the activity of ion transporters and/or channels. In particular, the Clomeleon construct can be used, for example, to determine halide ion concentrations, in particular, chloride. Once a cell line or transgenic animal has been engineered to express, for example, Clomeleon, either constitutively at varying levels or inducibly, the cell line or transgenic animal can be used in high-throughput screening methods that will identify modulators of ion transporters and/or channels, e.g., KCC2 and NKCC1.

The cell lines used to express the fluorescent ion detector, e.g., Clomeleon, can be selected as determined by one of skill in the art to be suitable for the screen. Although the screen identifies, for example, molecules that modulate the activity of ion transporters and channels, e.g., KCC2 and NKCC1, that are active in neurons, the cell lines chosen for the high-throughput screen can be any cell type sufficient to allow for the screen. It is possible, for example, that a cell line can be chosen that does not have ion transport activity, or a known ion transport activity, whereby the cell line can further be engineered to express one or more exogenous ion transport proteins and/or ion channels (e.g., KCC2 and/or NKCC1).

The high-throughput screen described herein can use, for example, a variety of mechanism to produce an ion challenge to the cell or transgenic animal that expresses the fluorescent ion detector. To ensure the integrity of the screen, however, the methods described herein utilize genetic constructs that can create an ion challenge. Genetic constructs that express, for example, halorhodopsin or the α1-glycine receptor (see Exemplification) are suitable for the methods described herein. These constructs inducibly provide a chloride challenge to the cellular environment.

As described herein, a high-throughput screen involves exposing a cell line or transgenic animal that expresses a fluorescent ion detector, e.g., Clomeleon, to a test agent. The exposed cell line or transgenic animal will then be challenged with an inducible Cl⁻ load, and the intracellular chloride ion concentration is determined using a fluorometric assay to detect, for example, YFP emissions. The intracellular chloride ion concentration is compared in the presence and absence of the test agent. A difference in the intracellular chloride ion concentration in the presence of the test agent versus the absence of the test agent indicates the test agent modulates the activity of one or more ion transporters or channels.

To identify a modulator that is specific to a particular transporter or ion channel, a cell line can be chosen with a known ability or lack thereof to transport, for example, chloride. The cell line can be engineered to express one or more specific ion transporters or channels, e.g., KCC2 or NKCC1. The result of the high-throughput screen described herein, then, would identify modulators of the particular transporter(s) or ion channel(s) that were engineered to be expressed in the particular cell line.

Also described herein are methods that identify general modulators of ion transport in a particular cell type. For example, to identify modulators of ion transport in, for example, neurons, the high-throughput screen can be used in neurons that express the fluorescent ion detector, e.g., Clomeleon.

If modulators are identified in cell lines, they can be validated, for example, in transgenic animals that allow for the quantifiable detection of intracellular ion concentrations, e.g., chloride. Although such transgenic animals can be used to screen and identify compounds as well, such animals are useful to validate the identified modulators in a more clinical in vivo environment.

The molecules identified as modulators of ion transport and/or channel activity can be active, for example, in any tissue where ion transport activity is necessary, e.g., the brain and kidneys.

### EXEMPLIFICATION

### Example 1

Unlike other ion transport systems such as the NaIK ATPase, there are no active chloride transporters. Cl⁻ homeostasis is driven indirectly by gradients generated by other active transport proteins. For Cl⁻ homeostasis, there are two major groups of transport proteins: the Na—K—Cl cotransporters (NKCC's) and the K—Cl cotransporters (KCC's). In neurons, the major transporters are NKCC1 and KCC2, with KCC2 being expressed only in neurons and in no other cell types. Both transporters set the amount of Cl⁻ inside the neuron and hence the reversal potential of Cl⁻ currents (ECI), which is the membrane potential at which Cl⁻ currents reverse or flip direction from inward to outward. The ECI value determines the direction of Cl⁻ currents passing through the GABA_{A} and glycine neurotransmitter receptors. When Cl⁻ currents are outward, they favor excitation, whereas when they are inward they favor inhibition.

As shown in FIG. 1, NKCC1 and KCC2 oppose one another. For example, in embryonic and neonatal neurons, NKCC1 dominates as KCC2 expression is low. Under these conditions the intracellular concentration of Cl⁻ is high, creating an ECI that is more positive than the membrane potential. This situation produces GABA_{A} currents that are normally outward and excitatory. In adult neurons there is a critical shift toward a system that is dominated by KCC2, creating low Cl⁻ concentrations inside the cell. This creates a reversed situation, where GABA_{A} currents are inward and inhibit brain activity. Without this KCC2-dependent inhibitory constraint on brain activity, neurons become dangerously hyperexcitable. In several neurological disorders KCC2 activity is drastically reduced, resulting in a buildup of Cl⁻ inside the cell and abnormal excitatory currents through the GABA_{A} receptors. In one embodiment, OCTA is used to discover and assay compounds that increase KCC2 activity to treat these widespread and debilitating disorders.

Clomeleon, a fusion protein that detects chloride, can be expressed in all mammalian cells including neurons. Described herein are cell lines, e.g., HEK293, that are modified, permanently, transiently or inducibly, to express Clomeleon protein. This cell line(s) described herein can also be modified to permanently, transiently or inducibly express KCC2 or NKCC1 protein. Unlike a Rb⁺ flux or thallium-based assay, OCTA does not utilize osmotic shock to challenge cation-chloride transporter function. OCTA challenges transporter activity by loading the cells with Cl⁻. To induce a Cl⁻ load, these cells permanently express one of two types of Cl⁻ channels: the α1-glycine receptor or halorhodopsin, which is activated by yellow light and conducts only Cl⁻ ions. In total, these cell lines will express Clomeleon, plus one cation-chloride transporter and one Cl− channel. Examples of cell lines include: NKCC1 lines with either α1-glycine receptor or halorhodopsin, and KCC2 lines with either α1-glycine receptor or halorhodopsin.

In parallel, OCTA technology will also utilize neurons obtained from genetically modified mice engineered to express Clomeleon alone, or Clomeleon and halorhodopsin. All neurons naturally respond to GABA, so there is no need to engineer animals to express GABAA or glycine receptors. We can then examine the effect of compounds that modulate Cl⁻ homeostasis in a physiological condition at various stages of development: in neonatal neurons when NKCC1 is at high levels and KCC2 at low levels, and in adult neurons when KCC2 is high and dominates the Cl⁻ homeostatic mechanism. These experiments can take on several forms including batch screening on isolated neurons, and rigorous examination of the compounds on individual neurons, slices of brain tissue, or in living mice. The more rigorous examination will utilize primarily conventional technology such as patch clamp rigs equipped with fluorescence microscopy.

### Example 2

### OCTA Technology in Cells and Tissue

OCTA technology is a fluorescence-based assay that measures the activity of cation-chloride transporters directly in real time. OCTA uses a genetically-encoded protein called Clomeleon that senses Cl⁻. Its properties depend on fluorescence resonance energy transfer (FRET), with yellow wavelength emission intensities that decrease with increasing Cl⁻ levels. Clomeleon can be expressed in all mammalian cells including neurons. In one embodiment, the assay utilizes an engineered cell line (HEK293) that is permanently transfected to express the Clomeleon fusion protein. This cell line can be modified to permanently express either exogenous KCC2 or exogenous NKCC1 protein. Unlike a Rb⁺ flux or thallium-based assay, OCTA does not utilize osmotic shock to challenge cation-chloride transporter function. OCTA challenges transporter activity by loading the cells with Cl⁻. To induce a Cl⁻ load, for example, these cells permanently express one of two types of Cl⁻ channels: the α1-glycine receptor or halorhodopsin.

The α1-glycine receptor is a vertebrate homopentameric neurotransmitter-gated ion channel that binds to the neurotransmitter glycine to open a Cl⁻ permeable pore (Keramidas, A. et al., Prog. Biophys. Mol. Biol., 86:161-204, 2004). Halorhodopsin is a bacterial protein activated by yellow light and conducts only Cl⁻ ions (Chow, B. et al., Prog. Brain Res., 196:49-61, 2012). The advantage that halorhodopsin provides is that the Cl⁻ load can be induced by light exposure, as opposed to utilizing a solution exchange system that is required for a glycine-induced Cl⁻ load. In total, these cell lines express Clomeleon, a cation-chloride transporter and a Cl⁻ channel, for a total of four cell lines: an NKCC1 line with either α1-glycine receptor or halorhodopsin, and a KCC2 cell line with either al-glycine receptor or halorhodopsin.

OCTA technology also utilizes neurons obtained from genetically modified mice engineered to express Clomeleon alone, or Clomeleon and halorhodopsin. All neurons naturally respond to GABA, so there is no need to engineer animals to express GABA_{A} or glycine receptors. The effect of test compounds are then determined to identify compounds that modulate Cl⁻ homeostasis at various stages of development: in neonatal neurons where NKCC1 is at high levels and KCC2 at low levels, and in adult neurons where KCC2 is at high levels and dominates the Cl⁻ homeostatic mechanism. These experiments allow for batch screening on isolated neurons and comparative examination of the compounds in individual neurons, slices of brain tissue, or in living mice using conventional technology such as patch clamp rigs equipped with fluorescence microscopy.

### Example 3

### OCTA Experiment

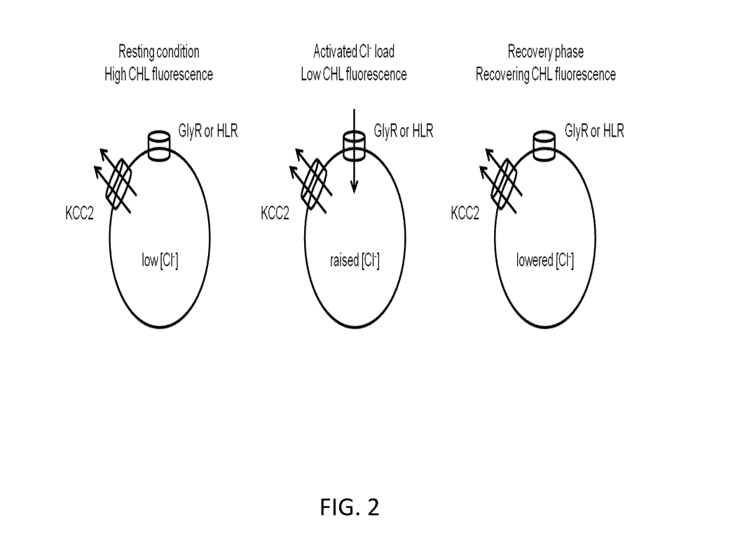

Cells expressing KCC2 or NKCC1 have intracellular Cl⁻ concentrations ranging between 5-40 mM Cl⁻, depending on relative transporter expression, or in the case of the OCTA cell lines whichever line is chosen for a given experiment. Clomeleon exhibits a baseline fluorescence that is quantified every 15 s over the course of 5 min. Cell lines are challenged with a Cl⁻ load by either exposing cells to glycine or yellow light depending on the cell line (with expressed glycine receptor or halorhodopsin), for 15-30 s (FIG. 2). Clomeleon measurements proceed as before. The rate at which the fluorescence returns to baseline levels indicates the level and rate of transporter activity. The same experiment optionally can be performed in a parallel set of cells in the presence of a test compound, or repeated on the same cells in the presence of the test compound. The entire experiment lasts no more than 25 min per test compound. The solutions required for these experiments are inexpensive and non-toxic electrophysiological saline solutions, e.g., 140 mM NaCl, 2 mM CaCl₂, 2 mM MgCl₂, 10 mM glucose, and 10 mM HEPES (pH buffered to 7.4 with NaOH).

### Example 4

### OCTA Experiment for Cultured Neurons

Adult neurons under basal conditions exhibit a strong Cl⁻ extrusion capacity that is dependent on KCC2 function. Mere activation of a high Cl⁻ conductance is typically insufficient to overwhelm Cl⁻ homeostasis to an extent that would enable characterization of compounds that increase KCC2 function. A greater challenge is therefore necessary. Glutamate exposure for short periods of time causes hyperactivation of the neuronal network that collapses the Cl⁻ gradient and inhibits KCC2 function. Thus unlike the cell line OCTA assay, cells only need to be exposed to glutamate to cause the Cl⁻ load. However, these neurons must be made to express Clomeleon to measure changes in intracellular Cl⁻.

### Example 5

### OCTA-Based Batch Screening Device

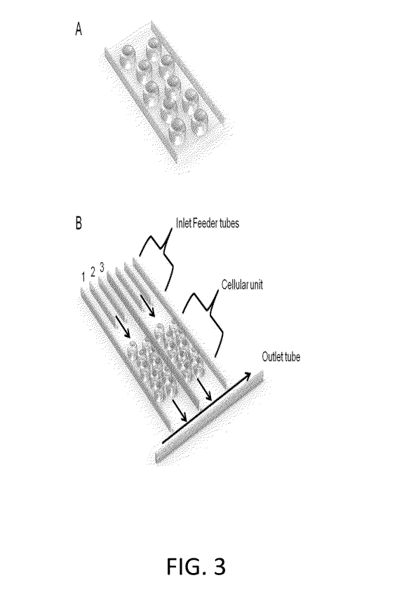

The batch screening device is based on a single unit chamber that is easily multiplied for industrial scale experiments. The chamber is made of clear plastic and is rectangular (5 mm wide, 10 mm long, 5 mm tall (FIG. 3A)). Cells or neurons are grown in these chambers until the time of the experiments. Each unit is attached to flow tubes, with an inlet on one end and an outlet on the other, this allows for rapid and complete solution exchange for application of the test compound, and glycine or GABA to activate a Cl⁻ load (if necessary—otherwise yellow light can be applied from above to activate halorhodopsin). An optical fluorescence imager and light source is then positioned above or below each unit to measure the fluorescence of Clomeleon. The imaging as well as the solution exchange can be automated. The cell chambers are removable and disposable such that cells, particularly neurons, can be grown in CO₂ incubators on the chambers. The chambers can be moved to the imaging area and fit into place (FIG. 3B). The outlet and inlet tubes are fixed to the imaging device. The outlet tube is for collecting waste and can be under modest vacuum pressure, whereas the inlet tubes can be under gravity pressure due to the wide dimensions. The inlet pipe is split into three separate tubes: one for control saline, one for glycine/GABA, one for the test compound. If necessary, more inlet tubes can be added accordingly. Each inlet tube is connected to a reservoir containing each solution and has a solenoid valve pinch-connector to allow for automated control of solution flow from each reservoir.

### Example 6

### Uses of Octa

1) The major therapeutic problem this invention will address is the lack of selective antagonists for NKCC1 and positive KCC2 modulators for the treatment of several neurological deficits including, autism spectrum disorders, depression, epilepsy, ischemia, status epilepticus, peripheral nerve damage, neurodegeneration, neuropathic pain, schizophrenia, seizures associated with brain tumors, temporal lobe epilepsy and traumatic brain injury.

2) A major technological problem with current ion detection methods is the lack of a robust screening method and device for cation-chloride transporters that works with cell lines and mammalian neurons.

The tried and proven method for measuring cation-chloride transporter activity is the rubidium (Rb⁺) flux assay. This is based on the ability of the transporter proteins to substitute Rb⁺ for K. Under physiological conditions, KCC2 co-transports one molecule of K⁺ and one molecule of Cl⁻ across the membrane. In this assay, Rb⁺ is substituted for K⁺, thus, KCC2 co-transports equal amounts of Rb⁺ and Cl⁻. To measure the movement of these ions, however, Rb⁺ is radiolabeled (⁸⁶Rb), and the sensors measure changes in the amount of radioactivity over the period of several minutes (Delpire, E. et al., Proc. Natl. Acad. Sci. USA, 106:5383:8, 2009).

A newer method is a thallium-based assay. Similarly, thallium can also be substituted for K⁺. In this case, fluorescent dyes that detect thallium measure the changes in fluorescence due to cation-chloride transporter activity (Zhang, D. et al., J. Biomol. Screen., 15:177-84, 2010).

a) OCTA overcomes the major methodological problem by which other screens activate or alter basal levels of cation-chloride transporter function to measure further changes caused by the compounds being screened. Altering the concentration of the substrate ions (e.g., Cl⁻ and K⁺ or Rb⁺) alters the amount of ions transported in either direction across the membrane. Current screening methods challenge cotransporter function by utilizing osmotic shock (hypo- or hypertonic solutions). While osmotic shock is akin to edema (cell swelling) it is secondary to the pathophysiological insult. Cell swelling is caused by the movement of ions such as K+ and Cl−, not vice versa. Hence, the condition utilized by these screens is incorrect and lethal to the cell and can therefore produce false positives and negatives. OCTA challenges the function of the cotransporters using conditions that directly mimic the pathophysiological insult and are far less lethal. By expressing a non-native Cl⁻ channel in the cells of interest (could also be endogenous Cl⁻ permeable glycine or GABA_{A} receptors if neurons are being examined); a Cl⁻ load that challenges cotransporter function is created. The ability of test compounds to modulate (increase or decrease) the response of the cotransporters to the Cl⁻ load challenge is measured directly by fluorescence imaging. OCTA is far simpler, more direct, and occurs in real time.

b) Another major problem of the Rb-flux and thallium-based assays is the lethality and toxicity of the substances to the cells, and the difficulties that technicians must endure to work with radioactive isotopes and toxic chemicals. OCTA does not utilize either type of compound. Nothing associated with OCTA is toxic or radioactive, thereby removing hazards technicians. Furthermore, cells can be examined for far longer periods due to the lack of toxicity of the compounds used in OCTA.

c) OCTA has the ability to examine cotransporter function in neurons. Unlike cell lines, neurons are the target tissue of interest and so they naturally possess the cellular machinery that test compounds interact with if they are used therapeutically. Furthermore, this machinery provides the potential to analyze how test compounds can manipulate the endogenous machinery that has already been proven to modulate cation-chloride transporter function. OCTA experiments in neurons, in combination with OCTA measurements in cell lines, allow for batch screening of test compounds on all of the cation-chloride transporters and the endogenous machinery that regulates their function to discover and characterize novel ligands with wide ranging therapeutic potential.

### Example 7

### Optical Cation-Chloride Transporter Assay (OCTA)

### Cell Line-Production

Permanently transfect human embryonic kidney (HEK) cells with a suitable vector for expressing Clomeleon.

Visually select Clomeleon-positive cells using fluorescence microscopy: transfected cells fluoresce under blue light, whereas negative cell have no fluorescence.

Maintain permanently transfected Clomeleon-positive (CHL) cell line in appropriate selection media.

Transfect Chinese hamster lung (CHL) cells with a suitable vector for expressing halorhodopsin.

Perform electrophysiological assays to select cells expressing halorhodopsin (CHL-HLR cells).

Maintain permanently transfected CHL-HLR cells in appropriate selection media.

Transfect CHL-HLR cells with KCC2 DNA (the same procedure is also used for NKCC1).

Visually select KCC2-positive cells using fluorescence microscopy: due to the function of KCC2, the fluorescence spectrum of Clomeleon is different compared to cells lacking KCC2.

Maintain CHL-HLR-KCC2 cells (OCTA cell line) in appropriate selection media; cells are ready to use for experiments.

### Neuron-Production

Cross halorhodopsin transgenic mice with Clomeleon transgenic mice to produce a double-transgenic animal (OCTA mice). KCC2 is an endogenous neuron-specific protein expressed by all mammals, so it is expressed in OCTA mice.

Use PCR screening to confirm genotypes of offspring.

Maintain mouse colony; animals are ready for use.

A Cl⁻ load can be induced by other agents due to the presence of endogenous Cl⁻ channels, and so only the Clomeleon line could be used if desired.

### OCTA Assay

Use dissected tissue from OCTA mice or cultured neurons prepared from OCTA mice; or use OCTA cell line.

Using a fluorescence microscope equipped with appropriate emission wavelengths (yellow for halorhodopsin activation; blue for Clomeleon activation), measure baseline Clomeleon emission spectra.

After establishing baseline values, induce a Cl⁻ load by activating halorhodopsin, this should cause a temporary reduction in the intensity of the Clomeleon emission spectra in the yellow wavelength range.

Alternatively in neurons, a Cl⁻ load is caused by exposing the cells to pathophysiological compounds such as glutamate, zero-Mg²⁺ solutions or furosemide (or other suitable compound as determined by one of skill in the art). Therefore the OCTA assays can be performed on neurons without the halorhodopsin transgene, thereby simplifying the procedure outlined in the “neuron-production” section. These experiments only require the Clomeleon transgenic mouse line. However, these experiments would require a solution exchange system that is fit onto the microscope, as would be known to one of skill in the art.

Take Clomeleon measurements at regular intervals over a predetermined time period.

Plot the recovery of the Clomeleon emission intensity against time to determine the rate of KCC2 function.

Repeat this procedure, or perform this procedure on a separate set of cells, in the presence of various test compounds to examine if they can increase the rate of recovery of Clomeleon emission intensity, which is indicative of an increase of KCC2 activity.

### Other Embodiments

Other embodiments will be evident to those of skill in the art. It should be understood that the foregoing detailed description is provided for clarity only and is merely exemplary. The spirit and scope of the present disclosure are not limited to the above examples, but are encompassed by the following claims. The contents of all references cited herein are incorporated by reference in their entireties.

What is claimed is:

1. A method of identifying a modulator of an ion transport protein or ion channel comprising,
a) contacting a cell line that expresses a fluorescent ion detector with a test agent;
b) creating a chloride challenge to the contacted cell line; and
c) determining the intracellular chloride ion concentration,
wherein an intracellular chloride ion concentration that is statistically different from a cell line not contacted with the test agent and exposed to the same chloride ion challenge indicates that the test compound is a modulator of an ion transport protein or ion channel.

2. The method of claim 1, wherein the ion channel/transporter is a chloride ion channel/transporter.

3. The method of claim 2, wherein the chloride ion channel/transporter is KCC2 or NKCC1.

4. The method of claim 1, wherein the fluorescent ion detector is Clomeleon.

5. The method of claim 1, wherein the identified modulator is further validated in a transgenic animal.

6. The method of claim 5, wherein the transgenic animal expresses a fluorescent ion detector.

7. The method of claim 6, wherein the fluorescent ion detector is Clomeleon.

8. A modulator of an ion transport protein or ion channel that is identified by the method of claim 1.

9. A method of treating a disease or disorder associated with aberrant intracellular ion concentrations comprising administering a therapeutically effective amount of a modulator identified by the method of claim 1.

10. An engineered cell line comprising:
a) a vector that expresses a fluorescent ion detection protein; and
b) a vector that expresses an exogenous ion transport protein or ion channel.

11. The cell line of claim 10, wherein the fluorescent ion detection protein is Clomeleon.

12. The cell line of claim 10, wherein the exogenous ion transport protein or ion channel is KCC2 or NKCC1.

13. The cell line of claim 10, wherein the cell line is a human embryonic kidney cell line, a Chinese hamster lung cell line, a COS-7 cell line or a fibroblast cell line.

14. A transgenic animal comprising a vector comprising a fluorescent ion detection protein.

15. The transgenic animal of claim 14, wherein the transgenic animal further comprises a vector that expresses an exogenous ion transport protein or ion channel.

16. The transgenic animal of claim 14, wherein the animal is a mammal.

17. The transgenic animal of claim 16, wherein the mammal is a rodent.
